(19) United States
(12) Patent Application Publication
(10) Pub. No.: US 2025/0054579 A1
(43) Pub. Date: Feb. 13, 2025

(54) ANALYSIS AND DETERMINATION OF POLYPEPTIDE SEQUENCES

(71) Applicant: George Mason University, Fairfax, VA (US)

(72) Inventors: Barney Bishop, Annandale, VA (US); Amy Carfagno, Triangle, VA (US); Madison Werkheiser, Madison, AL (US); Maria Emelianenko, Burke, VA (US); Sofia Jacik, Fairfax, VA (US); Gandhar Anirudha, Fairfax, VA (US)

(21) Appl. No.: 18/799,735

(22) Filed: Aug. 9, 2024

Related U.S. Application Data
(60) Provisional application No. 63/518,552, filed on Aug. 9, 2023.

(51) Int. Cl. G16B 30/20; C12Q 1/37; G01N 33/68; G06N 20/00; G16B 30/10

(52) U.S. Cl. CPC G16B 30/20; C12Q 1/37; G01N 33/6818; G01N 33/6848; G06N 20/00; G16B 30/10

(57) ABSTRACT

In aspects, a method includes: dividing a sample including a mixture of polypeptides into at least two factions that include a starting fraction and at least one other fraction; contacting polypeptides in each of the other fraction(s) with at least one agent for cleavage of the polypeptides, to provide at least one fragmented fraction; analyzing polypeptides in the starting fraction and polypeptides in the fragmented fraction(s) to provide starting sequences for the starting fraction and fragment sequences for the fragmented fraction(s); grouping the starting sequences and the fragment sequences into at least one cluster, where each of the cluster(s) include at least one starting sequence and at least one fragment sequence; and for each cluster, generating a computed sequence, based on values in the at least one starting sequence and values in the at least one fragment sequence of the respective cluster, for a polypeptide in the sample.

This application claims the benefit of and priority to U.S. Provisional Application No. 63/518,552, filed on Aug. 9, 2023, the entire contents of which are incorporated by reference herein.

### GOVERNMENT SUPPORT STATEMENT

This invention was made with government support under grant number 2131062 awarded by the National Science Foundation. The government has certain rights in the invention.

### TECHNICAL FIELD

The present disclosure is drawn to analyses and de novo elucidation of high-confidence peptide sequences (e.g., intact native peptide sequences) from complex mixtures of proteins. The provided methods and compositions include the use of an algorithm and program that combines data from parallel sequence analyses of polypeptide samples where the polypeptide samples, e.g., are intact native and compared to enzymatically or chemically cleaved polypeptides.

### BACKGROUND

Proteomics research focuses on large-scale studies to characterize the proteome, the entire set of proteins, in a living organism. Historically, it has been challenging to determine the sequences of larger intact native peptides from complex samples de novo from tandem mass spectra without the aid of genomic, transcriptomic and/or proteomic sequence data. Typically, identification of protein sequences utilizing liquid chromatography-tandem mass spectrometry (LC-MS/MS) is performed either through database-search methods (given that a set of predictive sequences for a certain organism is readily available) or de novo sequencing based on fragmentation data alone. While an essential step in the determination of novel peptide sequences, de novo sequencing often yields lower accuracies and less conclusive results compared to the former due to a lack of reference information. Accordingly, an improved method for de novo sequencing could be highly beneficial for the evaluation of organisms lacking suitable pre-existing sequence databases, allowing better identification of peptides of interest (e.g., antimicrobial peptides).

### SUMMARY

The present disclosure is drawn to methods and compositions for large scale analyses and elucidation of high-confidence peptide sequences (e.g., intact native peptide sequences) from complex mixtures of proteins. Said method enables one to generate a computed de novo polypeptide sequence. The terms “peptide” and “polypeptide” may be used interchangeably herein. The provided methods and compositions include the use of an algorithm and program that combines data from parallel analyses of polypeptide samples where starting polypeptide sequences (e.g., intact native polypeptide sequences) are compared to enzymatically or chemically cleaved polypeptide sequences wherein the polypeptide sequences are grouped into clusters with sequences aligned within the clusters. For each cluster of sequences, a computed sequence is then generated and may factor in potential parameters, such as for example, residue alignment between sequences, positional confidence scores, etc.

In aspects of the present disclosure, a polypeptide sequencing method includes: dividing a sample including a mixture of polypeptides into at least two factions, where the at least two fractions include a starting fraction and at least one other fraction; contacting polypeptides in each of the at least one other fraction with at least one agent for cleavage of the polypeptides within the at least one other fraction, to provide at least one fragmented fraction; analyzing polypeptides in the starting fraction and polypeptides in the at least one fragmented fraction to provide starting sequences for polypeptides in the starting fraction and fragment sequences for polypeptides in the at least one fragmented fraction; grouping the starting sequences and the fragment sequences into at least one cluster, where each of the at least one cluster includes at least one starting sequence of the starting sequences and at least one fragment sequence of the fragment sequences; and for each cluster of the at least one cluster, generating a computed sequence for the respective cluster based on values in the at least one starting sequence and values in the at least one fragment sequence of the respective cluster, where the computed sequence serves as a computationally determined sequence for a polypeptide in the sample.

In various embodiments of the method, the sample is derived from a source selected from the group consisting of: soil, water, air, agricultural, or industrial compositions.

In various embodiments of the method, the sample is a biological sample.

In various embodiments of the method, the biological sample is derived from an animal species with an immune system that produces molecules to defend against infections.

In various embodiments of the method, the mixture of polypeptides in the sample are enriched based on size, charge, or affinity chromatography.

In various embodiments of the method, the cleavage of the polypeptides is achieved enzymatically or chemically.

In various embodiments of the method, the at least one agent for cleavage of the polypeptides includes proteolytic enzymes.

In various embodiments of the method, the starting fraction includes intact native polypeptides.

In various embodiments of the method, the grouping the starting sequences and the fragment sequences into at least one cluster is based on applying at least one clustering criterion, where the at least one clustering criterion is applied to at least one of: comparison of two starting sequences, comparison of two fragment sequences, or comparison of a starting sequence with a fragment sequence.

In various embodiments of the method, the at least one clustering criterion includes at least one criterion using confidence scores for the starting sequences and confidences scores for the fragment sequences.

In various embodiments of the method, for each cluster of the at least one cluster, the generating the computed sequence for the respective cluster includes: positionally aligning the at least one starting sequence and the at least one fragment sequence of the respective cluster with each other, to provide aligned sequences having a plurality of aligned positions; and for each position of the plurality of aligned positions of the aligned sequences, determining a computed value for the respective position based on values of the aligned sequences at the respective position, where the computed values for the plurality of aligned positions form the computed sequence for the respective cluster.

In various embodiments of the method, the positionally aligning the at least one starting sequence and the at least one fragment sequence of the respective cluster with each other uses Gotoh local alignment technique and BLASTP scoring parameters.

In various embodiments of the method, the computed values for the plurality of aligned positions are consensus values.

In various embodiments of the method, the computed values for the plurality of aligned positions are determined further based on confidence scores for values of the aligned sequences at the plurality of aligned positions.

In various embodiments of the method, the method further includes, for each cluster of the at least one cluster: iterating, for at least one iteration, the generating the computed sequence for the respective cluster, where for each iteration, the generated computed sequence from the prior iteration is used as a starting sequence of the respective iteration.

In various embodiments of the method, the analyzing the polypeptides in the starting fraction and the polypeptides in the at least one the fragmented fraction includes analyzing the polypeptides in the starting fraction and the polypeptides in the at least one the fragmented fraction using tandem mass spectrometry.

In various embodiments of the method, the generating the computed sequence for the respective cluster includes generating the computed sequence for the respective cluster using a machine learning model.

In various embodiments of the method, the machine learning model is a sequence to sequence model.

In accordance with aspects of the present disclosure, a system is disclosed for polypeptide sequencing of a sample containing a mixture of polypeptides divided into at least two factions, where the at least two fractions include a starting fraction and at least one other fraction, and where polypeptides in each of the at least one other fraction are contacted with at least one agent for cleavage of the polypeptides within the at least one other fraction to provide at least one fragmented fraction. The system includes at least one processor, and at least one memory storing instructions which, when executed by the at least one processor, causes the system at least to perform any of the methods of the aforementioned paragraphs.

In accordance with aspects of the present disclosure, a processor-readable medium stores instructions for polypeptide sequencing of a sample containing a mixture of polypeptides divided into at least two factions, where the at least two fractions include a starting fraction and at least one other fraction, and where polypeptides in each of the at least one other fraction are contacted with at least one agent for cleavage of the polypeptides within the at least one other fraction to provide at least one fragmented fraction. The instructions, when executed by at least one processor of a system, cause the system to at least perform any of the methods of the aforementioned paragraphs.

The details of one or more embodiments of the disclosure are set forth in the accompanying drawings and the description below. Other features, objects, and advantages of the techniques described in this disclosure will be apparent from the description and drawings, and from the claims.

### BRIEF DESCRIPTION OF FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1 is a diagram of an example of an operation for generating starting sequences and fragment sequences of samples of polypeptides, in accordance with aspects of the present disclosure;

FIG. 2 is a diagram of an example of comparing and clustering starting sequences, in accordance with aspects of the present disclosure;

FIG. 3 is a diagram of an example of comparing and clustering fragment sequences, in accordance with aspects of the present disclosure;

FIG. 4 is a diagram of an example of an operation for aligning sequences in a cluster and generating a computed sequence, in accordance with aspects of the present disclosure;

FIG. 5 is a flow diagram of an operation for determining computed sequences for polypeptides in a sample, in accordance with aspects of the present disclosure;

FIG. 6 is a block diagram of computation components, in accordance with aspects of the present disclosure;

FIG. 7 is a diagram of example sequence operations for a first cluster of sequences, in accordance with aspects of the present disclosure; and

FIG. 8 is a diagram of example sequence operations for a second cluster of sequences, in accordance with aspects of the present disclosure.

### DETAILED DESCRIPTION

The present disclosure is drawn to methods and compositions for large scale analyses and elucidation of high-confidence polypeptide sequences (e.g., intact native computed de novo polypeptide sequences) from a complex mixture of polypeptides. The provided methods and compositions include the use of an algorithm and program that combines clustering and alignment data from parallel analyses of polypeptide samples where derived intact native polypeptide sequences are compared to derived enzymatically or chemically fragmented polypeptide sequences.

In the following description, certain specific details are set forth in order to provide a thorough understanding of disclosed aspects. However, one skilled in the relevant art will recognize that aspects may be practiced without one or more of these specific details or with other methods, components, materials, etc.

Reference throughout this specification to “one aspect” or “an aspect” means that a particular feature, structure, or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects.

As described below, a novel technology has been developed that combines data from parallel analyses of samples to elucidate higher confidence sequences of intact native peptides present in complex mixtures. The provided technology is unique, as the algorithm and associated software are usable for large-scale analyses and the elucidation of high-confidence intact native peptide sequences from complex mixtures.

The provided technology may be used for generating a computed de novo polypeptide sequence and may include: (i) dividing a sample comprising a mixture of polypeptides into multiple samples; designating one of the multiple samples an intact native fraction and designating the one or more additional multiple samples as fragmented fraction(s); (ii) contacting each of the one or more designated fragmented fractions with an agent for digestion of the polypeptides within the fragmented fraction(s); (iii) analysis of the intact native fraction polypeptides and the fragmented fraction of polypeptides using tandem mass spectrometry (MS/MS) for de novo determination of the peptide sequence of polypeptides in the intact native and fragmented fractions; (iv) grouping of peptide sequences from the different fractions based on clustering; (v) aligning the sequences within the clusters; and (vi) generating a computed sequence for each cluster thereby generating a computed de novo sequence.

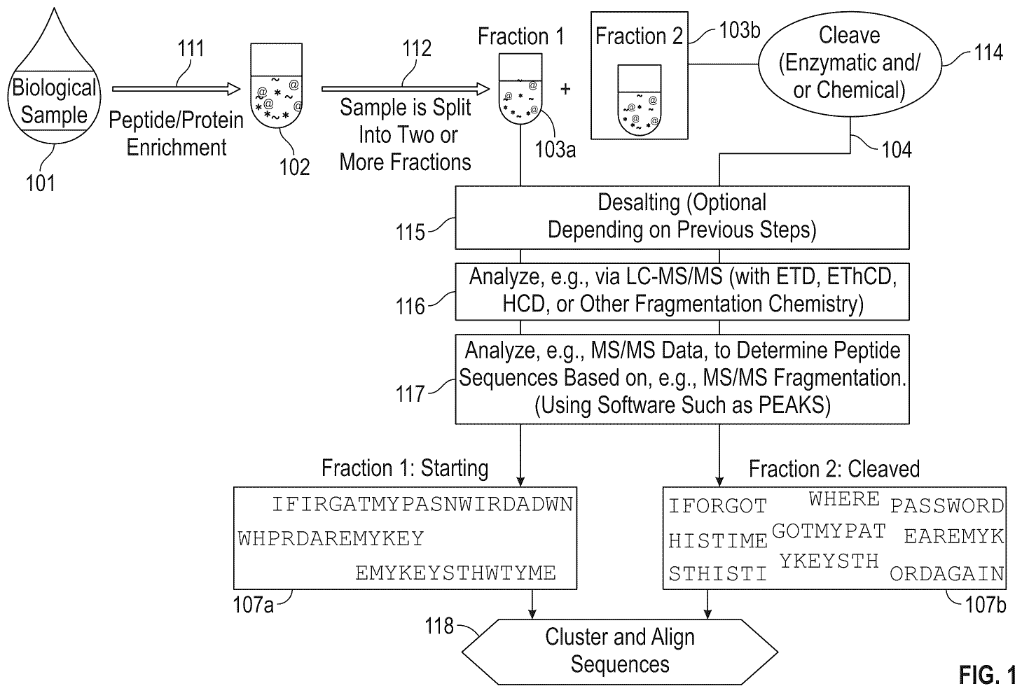

Referring to FIG. 1, there is shown an example of a polypeptide sequencing method. The provided polypeptide sequencing methods can be used to identify the polypeptide sequences of polypeptides found within a wide range of different sample types, including for example, soil, water, air, agricultural and industrial compositions. In one aspect, the sample is a biological sample which can be assayed to determine the polypeptide sequences of polypeptides within the sample. Such biological samples include extracts derived from cells, blood, or tissue samples derived from an organism. Such organisms include, but are not limited to, animals, plants, fungi, protozoa, bacteria, viruses and parasites. The presently provided sequencing method is particularly useful, for example, in sequencing of polypeptides within an organism lacking suitable pre-existing databases thereby allowing more efficient identification of peptide sequences of interest which have remained elusive due to their lack of abundance. Such polypeptides include, but are not limited to, antimicrobial or antiviral polypeptides.

In one specific non-limiting aspect, the sample 101 is derived from an animal species with an immune system that produces molecules to defend against infections, e.g., anti-microbials or antivirals. In one embodiment, said animal species includes various reptilian species and said provided methods may be used to identify novel antimicrobial and antiviral therapeutics. Said reptiles include, for example, the American alligator or Komodo dragon. In various embodiments, the animal species includes various amphibian species.

Methods for purification and enrichment 111 of particular classes of polypeptides from a sample 101 (e.g., particle harvesting, solid-phase extraction or precipitation, etc.) may optionally be utilized to decrease the complexity of the polypeptide sample prior to analysis. In certain embodiments, the proteins may be enriched based on size, charge, or affinity chromatography. In one example, an acetonitrile (ACN)-based extraction method may be used that depletes high abundance and high molecular weight proteins from a sample prior to mass spectrometric analysis. In a specific embodiment, polypeptide enrichment 111 may be accomplished using acetonitrile/formic acid precipitation. In a specific embodiment, an 80% acetonitrile/0.1% formic acid (FA) precipitation solvent and a 75% acetonitrile/0.1% FA precipitation solvent may be used to enrich for small molecular weight proteins.

Once the enriched polypeptide sample 102 is obtained, the sample 102 is divided (112) into two or more fractions 103a, 103b, which can be processed and analyzed in parallel. One fraction 103a is designated the starting fraction (which in some embodiments may be referred as an intact native fraction), and one or more additional fractions 103b are designated to become fragmented fraction(s) 104. The fragmented fractions 104 contain samples of the polypeptide mixture which are contacted with an agent for cleavage of the polypeptides within the sample, while the starting fraction 103a (e.g., intact native fraction) lacks contact with a cleavage agent.

For cleavage 114 of the polypeptide mixtures, such cleavage 114 may be achieved through the use of various different enzymes or chemicals. Proteolytic enzymes (proteases) that may be used for cleavage of polypeptide samples include, but are not limited to, trypsin, trypsin-LysC, chymotrypsin, Glu-C, papain, elastase, endoproteinase Arg-C, Endoproteinase Glu-C, Endoproteinase Lys-C, pepsin and carboxypeptidase. In an embodiment, the polypeptide sample 103b to be cleaved (to become the fragmented fraction 104) may be divided into one or more samples with each sample being proteolytically cleaved 114 using a different enzyme. The multiple enzymes recognize and specifically hydrolyze peptides at different positions in their sequences ideally yielding polypeptide fragments with overlapping sequences.

In one non-limiting illustration, the samples 103b are divided into two groups (not shown), with one being proteolyzed using trypsin and another with GluC. These two enzymes recognize and specifically hydrolyze peptides at different positions in their sequences, ideally yielding fragments with overlapping sequences. The proteolyzed peptide fragments 104 can then analyzed via, e.g., MS/MS.

As an alternative to proteolytic cleavage, the polypeptide samples 103b may be fragmented (114) using chemical-based cleavage. For example, cyanogen bromide (CNBr) cleaves at methionine (Met) residues; BNPS-skatole cleaves at tryptophan (Trp) residues; formic acid cleaves at aspartic acid-proline (Asp-Pro) peptide bonds; hydroxylamine cleaves at asparagine-glycine (Asn-Gly) peptide bonds, and 2-nitro-5-thiocyanobenzoic acid (NTCB) cleaves at cysteine (Cys) residues.

Following the cleavage 114 of sample polypeptides 103b, the cleaved samples 104 together with the starting fraction 103a (e.g., intact native sample), may optionally undergo desalting 115, and are then analyzed via, e.g., tandem mass spectrometry (MS/MS), nanopore technology, and/or Edman degradation sequencing, among other techniques 116. MS/MS will be used as an example of block 116 herein, but it will be understood that other analysis techniques may be employed at block 116. Accordingly, any disclosure that employs MS/MS shall be treated as though the disclosure employs other analysis techniques, as well. The tandem MS/MS 116 may be conducted in conjunction with electron-transfer dissociation (ETD), electron-transfer/higher-energy collisional dissociation (EThCD), higher-energy collisional dissociation (HCD) or other fragmentation chemistry which those skilled in the art will recognize. The MS/MS data are then analyzed 117 to assign amino acid sequences de novo to polypeptides in the starting (e.g., intact native) fraction 103a and assign amino acid sequences to polypeptides in the fragmented fraction 104. The sequences for the starting fraction 104 may be referred to herein as starting sequences 107a, and the sequences for the fragmented fraction 104 may be referred to herein as the fragment sequences 107b. Determination of the sequences 107a, 107b may be accomplished based on the MS/MS output using, for example, software such as PEAKS® provided by Bioinformatics Solutions Inc., among other possible software. Such analysis 117 may also provide average and positional confidence scores for the assigned sequences 107a, 107b and/or for individual amino acids in the sequences 107a, 107b based on how well the sequences are supported by the MS/MS spectral data. In accordance with aspects of the present disclosure, in blocks 116 and 117, providing the sequences based on MS/MS data may provide more accurate sequences for shorter sequences than for longer sequences and/or may provide higher confidence for shorter sequences than for longer sequences.

The example of FIG. 1 provides fictitious starting sequences 107a and fictitious fragment sequences 107b, which are shown in FIG. 1 and are as follows.

### Starting Sequences 107a

IFIRGATMYPASNWIRDADWN
WHPRDAREMYKEY
EMYKEYSTHWTYME

### Fragment Sequences 107b

IFORGOT
WHERE
PASSWORD
HISTIME
GOTMYPAT
EAREMYK
STHISTI
YKEYSTH
ORDAGAIN

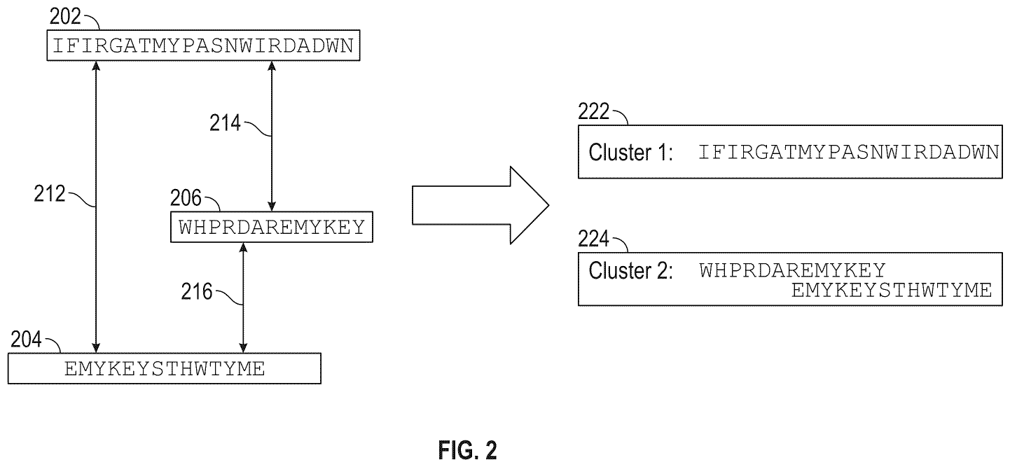

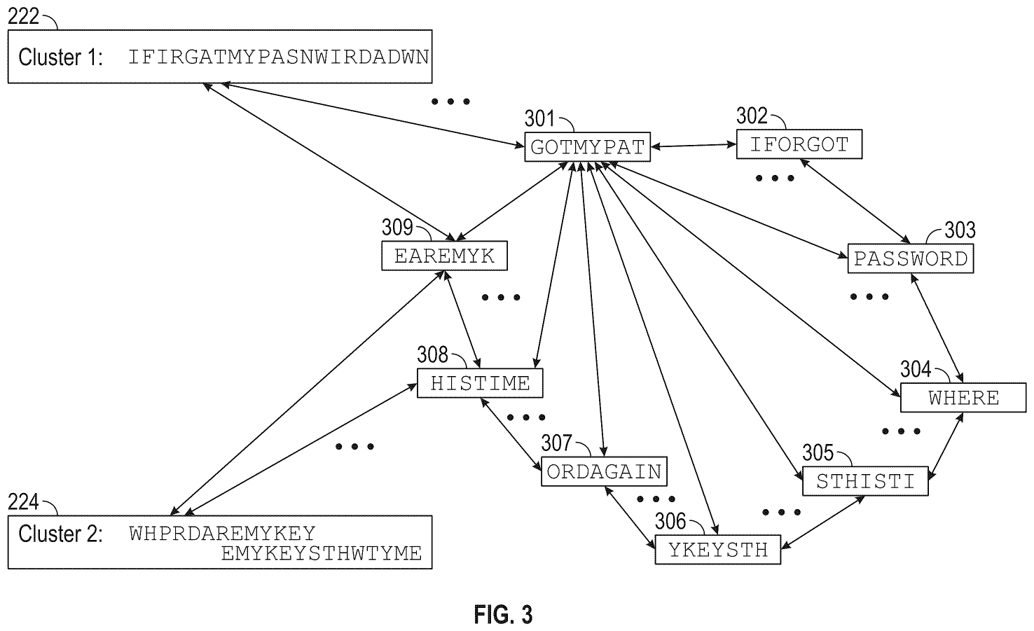

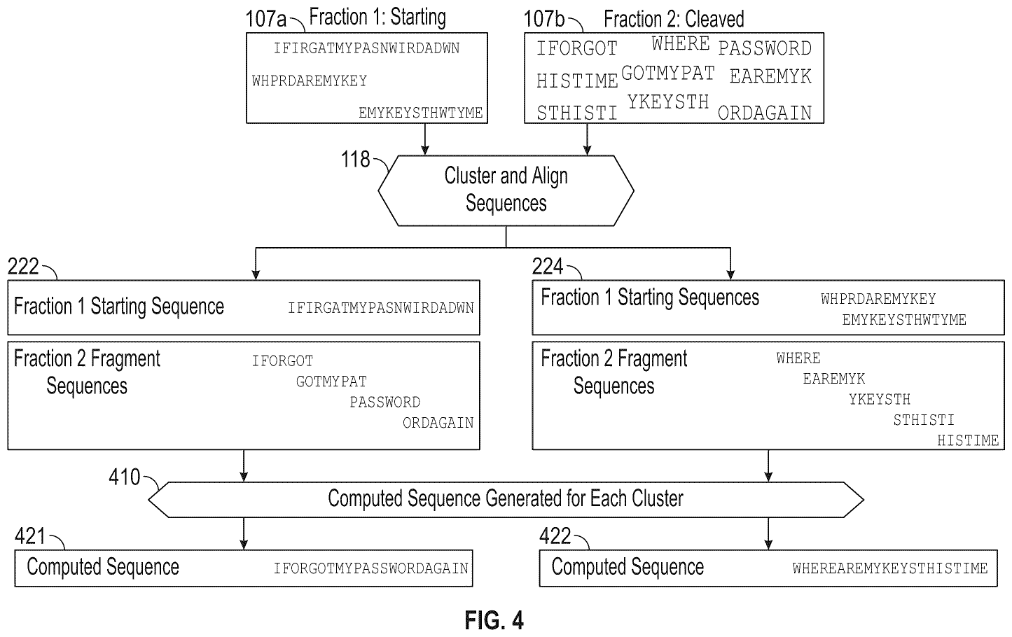

The starting sequences 107a and the fragment sequences 107b are clustered and aligned 118, which will be described in more detail in connection with FIG. 2 and FIG. 3. For now, it is sufficient to note that the starting sequences 107a and fragment sequences 107b are grouped into one or more clusters, where each cluster includes starting sequences and fragment sequences that are computationally estimated to be sequences for the same polypeptide. The sequences in each cluster are computationally aligned 118 with each other, and based on the alignment of the sequences in each cluster, a computed sequence for each cluster is determined, which will be described in connection with FIG. 4.

FIG. 1 is merely illustrative and variations are contemplated to be within the scope of the present disclosure. For example, in various embodiments, other components or processes may be incorporated into the operation of FIG. 1. In various embodiments, the operation of FIG. 1 may not include all of the components or processes shown in FIG. 1. Such and other variations are contemplated to be within the scope of the present disclosure.

FIG. 2 is a diagram showing a process for grouping starting sequences into clusters. Continuing with the example of FIG. 1, the fictitious starting sequences 107a include three sequences: IFIRGATMYPASNWIRDADWN, WHPRDAREMYKEY, and EMYKEYSTHWTYME. The grouping process operates to computationally estimate whether these sequences are all part of the same starting polypeptide or whether certain sequences are for different polypeptides.

In aspects of the present disclosure, the grouping process may perform a pairwise comparison between each pair of sequences. In the example, the pairwise comparisons include three comparisons—a first comparison 212, a second comparison 214, and a third comparison 216. In various embodiments, the pairwise comparisons may determine the degree of overlap between two sequences. In various embodiments, two sequences may overlap completely such that a longer sequence encompasses a shorter sequence. In various embodiments, two sequences may overlap partially such that a beginning or ending portion of one sequence does not overlap with the other sequence.

In the first comparison 212, the two sequences 202, 204 have several single-position overlaps (e.g., S, T, W, etc.) and have a two-position overlap (i.e., MY). In the illustrated example, for the two sequences 202, 204 having length 21 and length 14, a two-position overlap is not sufficient to group the two sequences 202, 204 into the same cluster. In the second comparison 214, the two sequences 202, 206 have several single-position overlaps (e.g., A, P, R, W, etc.) and have a three-position overlap (i.e., RDA). In the illustrated example, for the two sequences 202, 206 having length 21 and length 13, the three-position overlap is not sufficient to group the two sequences 202, 206 into the same cluster. In the third comparison 216, the two sequences 204, 206 have several single-position overlaps and have a five-position overlap (i.e., EMYKEY) formed by a beginning portion of sequence 206 and an end portion of sequence 206. In the illustrated example, for the two sequences 204, 206 having length 14 and length 13, the five-position partial overlap is sufficient to group the two sequences 204, 206 into the same cluster. As shown in FIG. 2, based on the three comparisons 212, 214, 216, the starting sequence 202 is in its own cluster 222, while the two starting sequences 204, 206 are grouped into a separate cluster 224.

The clustering criteria for how much overlap is sufficient to group two sequences into the same cluster can be configured in various ways. For example, in various embodiments, if the two sequences have lengths L₁ and L₂ and if the overlap is continuous and has a length of at least min (L₁,L₂)/3, the two sequences may be grouped into the same cluster. In various embodiments, an 3 overlap may be required to have minimum length, such as a three or another number. In various embodiments, the overlapping portion of two sequences may be non-continuous. For example, two sequences AABAA and AACAA have a four-character overlap that is non-continuous, such that the overlapping characters are separated by one or more non-overlapping characters. A non-continuous overlap may be sufficient to group the two sequences into the same cluster. In various embodiments, in a non-continuous overlap, there may be a criterion that overlapping characters may only be separated from each other by a maximum number of non-overlapping characters, such as separated by only one non-overlapping character, or another number. In various embodiments, different clustering criteria may be applied for grouping two sequences having complete overlap than for grouping two sequences having partial overlap. In various embodiments, different clustering criteria may be applied for grouping two sequences having continuous overlap than for grouping two sequences having non-continuous overlap. Such and other clustering criteria for grouping two sequences into the same cluster are contemplated to be within the scope of the present disclosure.

In various embodiments, each of the sequences may be accompanied by confidence scores for the sequences and/or confidence scores for individual positions in the sequences. For example, when the analysis 117 of FIG. 1 uses the PEAKS® software, the output of the software may include confidence scores for individual positions in the sequences. As mentioned above, providing the sequences based on MS/MS data may provide more accurate sequences for shorter sequences than for longer sequences and/or may provide higher confidence scores for shorter sequences than for longer sequences.

In accordance with aspects of the present disclosure, when confidence scores are available for sequences, they may be used in the clustering criteria for grouping sequences into clusters. In various embodiments, one or more thresholds may be used in connection with the confidence scores. For example, if two sequences have a sufficient overlap length (e.g., overlap length above a length threshold), but one or both confidence scores are below a score threshold, the two sequences may not be grouped into the same cluster. In various embodiments, where individual position scores are available, there may be used in the clustering criteria for grouping sequences into clusters. In various embodiments, clustering criteria may require that each overlapping position has a position confidence score above a score threshold. In various embodiments, clustering criteria may require that a certain percentage or certain number of overlapping positions have a position confidence score above a score threshold. In various embodiments, confidence scores for sequences as well as confidence scores for positions in a sequence may both be used in clustering criteria. Such and other embodiments are contemplated to be within the scope of the present disclosure.

The result of the grouping process of FIG. 2 is a computational estimate that the sequence(s) in each of the clusters 222, 224 are sequence(s) for the same starting polypeptide. Accordingly, in the illustrated example, the process computationally estimated that cluster 222 and cluster 224 correspond to two separate starting polypeptides in the starting fraction 107a. Although not shown, in various embodiments, a starting sequence may be included in more than one cluster. Below, the computational estimates for grouping fragment sequences (e.g., 107b, FIG. 1) will now be addressed in connection with FIG. 3.

FIG. 3 is a diagram showing a process for grouping fragment sequences into clusters, including into the clusters 222, 224 determined by the process of FIG. 2, and/or possibly into clusters different from the clusters 222, 224. Continuing with the example of FIG. 1, the fictitious fragment sequences 107b include nine sequences: GOTMYPAT, IFORGOT, PASSWORD, WHERE, STHISTI, YKEYSTH, ORDAGAIN, HISTIME, and EAREMYK. The grouping process operates to computationally estimate whether these sequences are part of the same starting polypeptide or whether certain sequences are part of different starting polypeptides.

In aspects of the present disclosure, the grouping process may perform a pairwise comparison between each pair of fragment sequences as well as between each pair of starting sequences and fragment sequences. In the example, the pairwise comparisons between the nine fragment sequences 301-309 include thirty-six comparisons, and the pairwise comparisons between the nine fragment sequences 301-309 and the three starting sequences 202, 204, 206 include twenty-seven comparisons.

As mentioned above in connection with FIG. 2, two sequences may overlap completely such that a longer sequence encompasses a shorter sequence, or two sequences may overlap partially such that a beginning or ending portion of one sequence does not overlap with the other sequence. There are many possible clustering criteria for grouping two sequences into the same cluster, including the clustering criteria described in connection with FIG. 2. In various embodiments, different clustering criteria may be applied to a comparison of two fragment sequences than clustering criteria applied to a comparison of a starting sequence with a fragment sequence. All such clustering criteria are contemplated to be within the scope of the present disclosure.

Continuing with the example, and to describe some of the sixty-three comparisons, the pairwise comparison of fragment sequence 305 (STHISTI) and fragment sequence 308 (HISTIME) has a five-position overlap (HISTI) for the two sequences of length 7, which in the example is sufficient to group the two fragment sequences 305, 308 into the same cluster. Comparing the same two fragment sequences 305, 308 to the first cluster 222 and second cluster 224, fragment sequence 305 overlaps with the first cluster 222 by just single positions and overlaps with the second cluster 224 by a three-position continuous overlap (i.e., STH). Fragment sequence 308 overlaps with the first cluster 222 by just single positions and overlaps with the second cluster 224 by a three-position non-continuous overlap (i.e., T_ME). Because both fragment sequences 305, 308 are clustered together, and both have greater overlap with the second cluster 224 than with the first cluster 222, and the overlap length is three positions for both fragment sequences 305, 308 with the second cluster 224, this is sufficient information to group the fragment sequences 305, 308 into the second cluster 224. The other pairwise comparisons are performed in the same manner to group the fragment sequences 301-309 into clusters. As mentioned above, if confidence scores for sequences and/or confidence scores for positions in sequences are available, such scores may be used in the clustering criteria and may be used with one or more thresholds.

The results of the comparisons is that fragment sequences 301, 302, 303, and 307 are grouped into the first cluster 222, and fragment sequences 304, 305, 306, 308, and 309 are grouped into the second cluster 224, which is shown in FIG. 4. Although not shown, in various embodiments, a fragment sequence may be included in more than one cluster. The following will describe, with respect to FIG. 4, the alignment aspect of operation 118 and further operations.

As mentioned above, the sequences in a cluster are grouped because they were computationally estimated to be sequences for the same polypeptide. In accordance with aspects of the present disclosure, the information in the sequences of a cluster can be analyzed to generate a computed sequence that serves as an estimate of the true sequence of the polypeptide corresponding to the cluster.

FIG. 4 is a diagram of an operation for aligning sequences in a cluster and analyzing similarities and differences at each position of the aligned sequences. The starting sequences 107a, the fragment sequences 107b, and the clustering aspect of operation 118 were described above in connection with FIG. 2 and FIG. 3. The result is a first cluster 222 with the following starting sequence and fragment sequences. (The second cluster 224 will be addressed farther below.)

### First Cluster

### Starting Sequence

IFIRGATMYPASNWIRDADWN

### Fragment Sequences

IFORGOT
PASSWORD
GOTMYPAT
ORDAGAIN

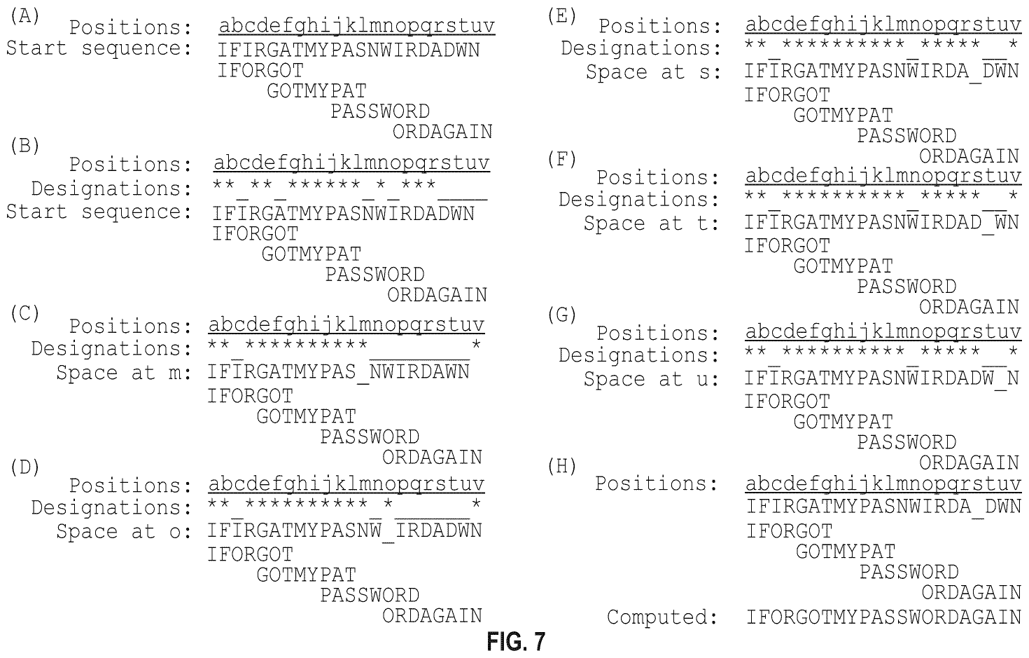

Based on the overlapping values between the sequences, the sequences can be aligned with each other as shown in FIG. 7 under label (A), where each position of the alignment is designated by a lower case letter. In various embodiments, the alignment operation can use the Gotoh local alignment technique and/or BLASTP scoring parameters, which persons skilled in the art will understand.

Notably, based on the alignment, the aligned fragment sequences show a total of 22 positions (positions a through v), while the starting sequence only has 21 positions. This difference of one position indicates that, more likely than not, the starting sequence may be missing a position (rather than the fragment sequences having an extraneous position). This is because, as mentioned above, providing the sequences based on MS/MS data may provide more accurate sequences for shorter sequences than for longer sequences and/or may provide higher confidence scores for shorter sequences than for longer sequences.

In various embodiments, when the length of the starting sequence is different form the length of the aligned fragment sequences, an approach for determining the location of the missing position in the starting sequence may start by marking positions as possible missing positions or as possible non-missing positions using various criteria. Using the same example, positions that may possibly be a non-missing position may include positions that have a starting sequence value that matches at least one fragment sequence value in the same position (e.g., positions a, b, d, etc.). Other criteria are contemplated. Based on such criteria, the markings for such positions are shown in FIG. 7 under label (B), where the * symbol is used to mark positions that are possible non-missing positions and the underscore symbol is used to mark positions that are possible missing positions.

In various embodiments, after marking the positions, a specific position can be identified where the specific position is marked as a possible missing position, and positions after the specific position contain more marked possible missing positions than marked possible non-missing positions. In the example, the specific position fitting such criteria is position m, which is a marked as a possible missing position, and after position m, there are more marked possible missing positions (five) than marked possible non-missing positions (four). Then, a placeholder value (such as a null/empty value or a space value, etc.) can be inserted into position m in the starting sequence to shift the remaining positions in the starting sequence by one position, and the result can be evaluated to determine whether it provides fewer marked possible missing positions. In the example of FIG. 7, inserting a space into position m does not result in fewer marked possible missing positions, as shown in FIG. 7 under label (C). The same process can be performed for each marked possible missing position after position m; that is, performed for positions o, s, t, and u, as shown in FIG. 7 under labels (D), (E), (F), and (G).

As shown by the examples in FIG. 7, inserting a space at positions m or o does not result in fewer marked possible missing position, while inserting a space at any of positions s, t, or u, results in one fewer marked possible missing position. Accordingly, any of positions s, t, or u could be a missing position in the starting sequence. In various embodiments, where confidence scores for individual positions are available, the confidence scores may be used to estimate the location of a missing position in the starting sequence. For example, if one of positions s, t, or u has the lowest confidence score, such position may be identified as the missing position based on having the lowest confidence score among them. The approach and criteria described above for identifying a missing position in a starting sequence are merely an example. Other approaches and criteria are contemplated for identifying a missing position in a starting sequence, and such approaches and criteria are within the scope of the present disclosure. Position s is used as the location of the missing position in the starting sequence, and the example is further described below.

In accordance with aspects of the present disclosure, each of the aligned sequences at each position is analyzed to determine a computed value for that position. In various embodiments, a computed value may be the value that has more occurrences than other values in the position, which may be referred to as a “consensus” value. For example, in the first cluster 222, position f has three values from the various sequences: A, O, and O. Because O has more occurrences than A, the consensus value for position f would be O. In various embodiments, if a position has only one value from the starting sequence and one value from a fragment sequence (such as position c), the value from the fragment sequence can be selected as the computed value for the position. In various embodiments, if a position has different values from all sequences, the value from the shortest sequence may be selected as the computed value. In various embodiments, when confidence scores for each position of each sequence are available, the confidence scores can be considered in determining the consensus value. For example, continuing with position c as an example, suppose value I has a confidence score of 0.8, and value O has a confidence score of 0.9. Because the confidence score for value O is higher than the confidence score for the value I, the computed value for position c may be selected to be O based on the confidence scores.

Determining the computed values at each position, the fictitious computed sequence 421 is determined to be IFORGOTMYPASSWORDAGAIN, as shown in FIG. 7 under label (H), which corrects the errors in the starting sequence IFIRGATMYPASNWIRDADWN.

In various embodiments, after determining the computed sequence 421, the alignment process of operation 118 and the computed sequence generation 410 can be iterated again for the first cluster 222 using the computed sequence 421 as the starting sequence. In various embodiments, any total number of iterations of the alignment process of operation 118 and the computed sequence generation 410 can be performed, such as one iteration, two iterations, or more than two iterations.

Using the same process for the second cluster 224, the second cluster 224 includes the following starting sequences and fragment sequences.

### Second Cluster

### Starting Sequences

WHPRDAREMYKEY
EMYKEYSTHWTYME

### Fragment Sequences

WHERE
EAREMYK
YKEYSTH
STHISTI
HISTIME

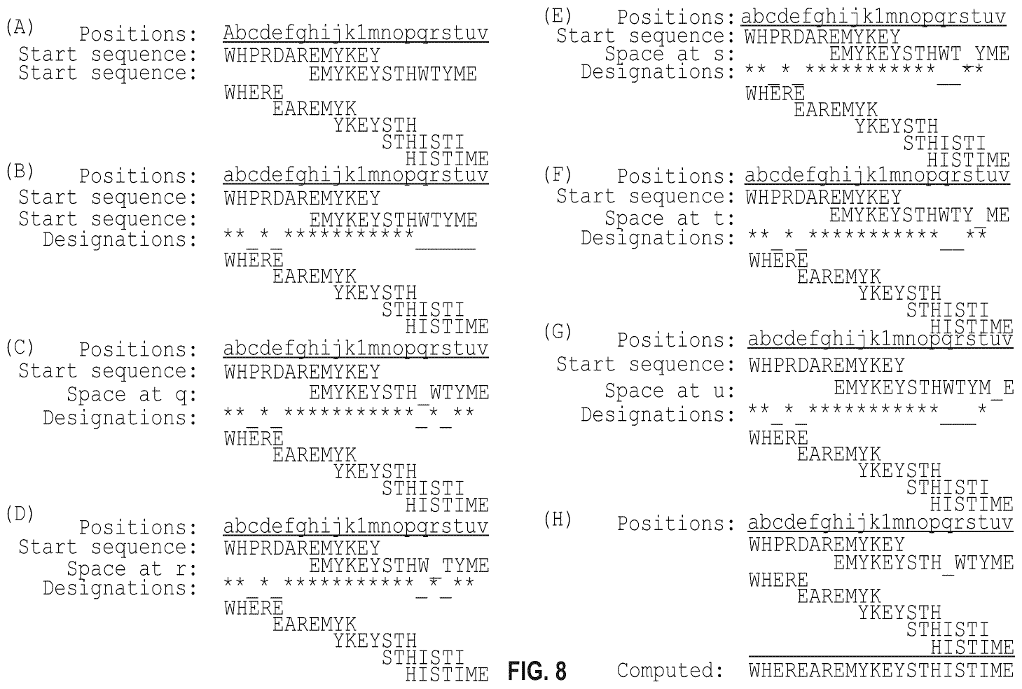

Based on the overlapping values between the sequences, they can be aligned with each other as shown in FIG. 8 under label (A), where each position of the alignment is designated by a lower case letter. In various embodiments, the alignment operation can use the Gotoh local alignment technique and/or BLASTP scoring parameters, which persons skilled in the art will understand.

Based on the alignment, the aligned fragment sequences show a total of 22 positions (positions a through v), while the starting sequences together only have 21 positions. This difference of one position indicates that, more likely than not, the starting sequence may be missing a position (rather than the fragment sequences having an extraneous position). This is because, as mentioned above, providing the sequences based on MS/MS data may provide more accurate sequences for shorter sequences than for longer sequences and/or may provide higher confidence scores for shorter sequences than for longer sequences.

The marking of possible missing positions and possible non-missing positions is shown in FIG. 8 under label (B). As in the example of the first cluster, positions that may possibly be a non-missing position may include positions that have a starting sequence value that matches at least one fragment sequence value in the same position. Other criteria are contemplated. Based on such criteria, the markings for such positions are shown below, where the * symbol is used to mark positions that are possible non-missing positions and the underscore symbol is used to mark positions that are possible missing positions.

In the example, position q is marked as a possible missing position, and after position q, there are more marked possible missing positions (five) than marked possible non-missing positions (zero), as shown in FIG. 8 under label (C). A placeholder value (such as a null/empty value or a space value, etc.) can be inserted into position q in the starting sequence to shift the remaining positions in the starting sequence by one position, and the result can be evaluated to determine whether it provides fewer marked possible missing positions. In the example below, inserting a space into position q results in three fewer marked possible missing positions. The same process can be performed for each marked possible missing position after position q; that is, performed for positions r, s, t, and u, as shown in FIG. 8 under labels (D), (E), (F), and (G).

As shown by the examples in FIG. 8, inserting a space at position u results in one fewer marked possible missing position, inserting a space at positions s or t results in two fewer marked possible missing positions, and inserting a space at positions q or r, results in three fewer marked possible missing positions. Therefore, positions q or r could be a missing position in the starting sequence. In various embodiments, where confidence scores for individual positions are available, the confidence scores may be used to estimate the location of a missing position in the starting sequence. For example, if one of positions q or r has the lower confidence score, such position may be identified as the missing position in the starting sequence based on having the lower confidence score between them.

Using position q as the location of the missing position in the starting sequence, and determining the computed values at each position, the fictitious computed sequence 422 is determined to be WHEREAREMYKEYSTHISTIME, as shown in FIG. 8 under label (H), which corrects the errors in the starting sequences WHPRDAREMYKEY and EMYKEYSTHWTYME.

In various embodiments, after determining the computed sequence 422, the alignment process of operation 118 and the computed sequence generation 410 can be iterated again for the second cluster 224 using the computed sequence 422 as the starting sequence. In various embodiments, any total number of iterations of the alignment process of operation 118 and the computed sequence generation 410 can be performed, such as one iteration, two iterations, or more than two iterations.

The examples described above in connection with FIG. 4, FIG. 7, and FIG. 8 are merely illustrative of the alignment aspect of operation 118 and of the computed sequence operation 410. Other approaches for the alignment aspect and for the computed sequence operation are contemplated to be within the scope of the present disclosure.

In aspects of the present disclosure, the computed sequences may be validated based on consistency with raw data (i.e., fragment ions supporting residue corrections made) and based on BLAST results against the sample organism's genome or transcriptome (if available)

The foregoing disclosure described an approach for computing an amino acid sequence for a polypeptide using clustering, alignment, and computational techniques. Other approaches are contemplated to be within the present disclosure, such as machine learning approaches.

In accordance with aspects of the present disclosure, various machine learning models may be used to provide a computed sequence based on receiving one or more starting sequences and/or one or more fragment sequences. An example of a machine learning model for such task is a sequence to sequence model, which receives an input sequence and provides an output sequence. A sequence to sequence model includes an encoder and a decoder. The encoder processes the input sequence to provide a vector, and the decoder processes the vector to provide an output sequence. The encoder and the decoder can each include a recurrent neural network (RNN) and long short-term memory (LSTM). Persons skilled in the art will understand how to implement sequence to sequence models. For training the sequence to sequence model, the input sequence(s) can be starting sequences and/or fragment sequences having known ground truth sequences, and the model can be trained so that the output sequences match the ground truth sequences as closely as possible.

The sequence to sequence model is merely an example, and other machine learning models are contemplated to be within the scope of the present disclosure. In various embodiments, the machine learning model(s) may be applied to one or more starting sequences and/or fragment sequences that have not been clustered. In various embodiments, the machine learning model(s) may be applied to one or more starting sequences and/or fragment sequences that have been clustered and that belong to the same cluster.

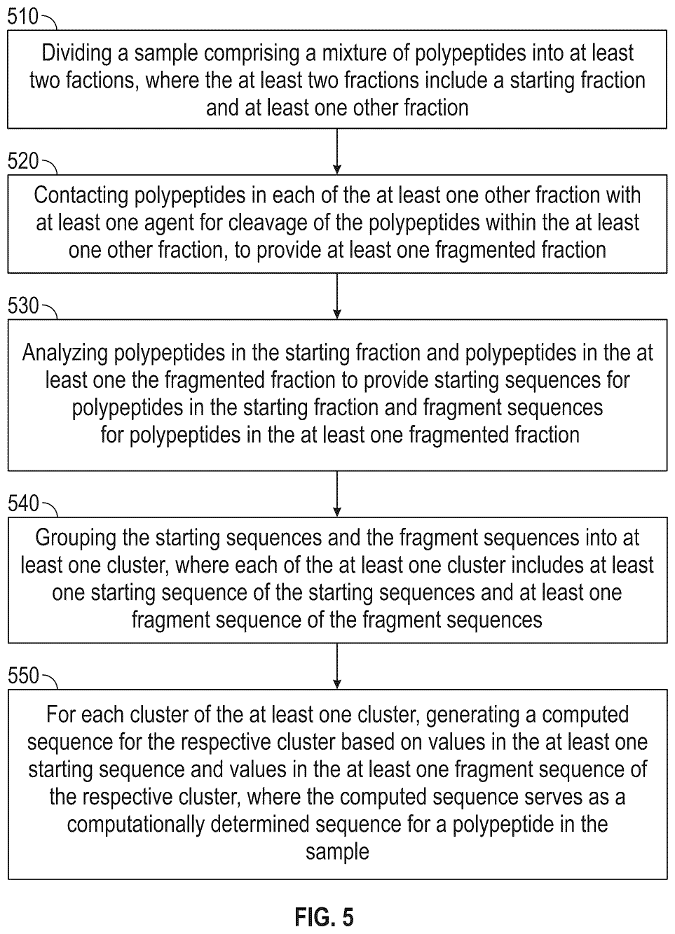

Referring now to FIG. 5, there is shown a flow diagram of an operation for determining computed sequences for polypeptides in a sample.

At block 510, the operation involves dividing a sample comprising a mixture of polypeptides into at least two factions, where the at least two fractions include a starting fraction and at least one other fraction. The starting fraction includes polypeptides that may be intact native polypeptides. The at least one other fraction includes polypeptides to be cleaved.

At block 520, the operation involves contacting polypeptides in each of the at least one other fraction with at least one agent for cleavage of the polypeptides within the at least one other fraction, to provide at least one fragmented fraction. The at least one agent may perform the cleavage enzymatically or chemically. The result of the cleavage is that the fragmented fraction(s) include fragments of the polypeptides in the starting fraction.

At block 530, the operation involves analyzing polypeptides in the starting fraction and polypeptides in the at least one the fragmented fraction based on tandem mass spectrometry (MS/MS) to provide starting sequences for polypeptides in the starting fraction and fragment sequences for polypeptides in the at least one fragmented fraction. The MS/MS provides data that may be used by software such as PEAKS® to provide an amino acid sequence for the polypeptides and may be used to provide confidence scores for the amino acid sequences and/or confidence scores for individual positions in the amino acid sequences. The sequences for the polypeptides in the starting frequence may be sequences for intact native polypeptides. The sequences for the polypeptides in the fragmented fraction(s) may be sequences for fragments of the native polypeptides.

At block 540, the operation involves grouping the starting sequences and the fragment sequences into at least one cluster, where each of the at least one cluster includes at least one starting sequence of the starting sequences and at least one fragment sequence of the fragment sequences. The grouping of sequences into clusters may be based on one or more clustering criterion. The clustering criteria may use confidence scores if they are available.

At block 550, the operation involves, for each cluster of the at least one cluster, generating a computed sequence for the respective cluster based on values in the at least one starting sequence and values in the at least one fragment sequence of the respective cluster, where the computed sequence serves as a computationally determined sequence for a polypeptide in the sample. The computed sequence may be a consensus sequence for an intact native polypeptide. In various embodiments, generating the computed sequence involves positionally aligning the at least one starting sequence and the at least one fragment sequence of a cluster with each other, to provide aligned sequences having a plurality of aligned positions, and for each position of the plurality of aligned positions of the aligned sequences, determining a computed value for the respective position based on values of the aligned sequences at the respective position. Then, the computed values for the plurality of aligned positions form the computed sequence for the cluster.

FIG. 5 and the description above are merely an example and variations are contemplated to be within the scope of the present disclosure. In various embodiments, the operation may include other blocks not shown or described in connection with FIG. 5. In various embodiments, the operation may not include every block shown or described in connection with FIG. 5. In various embodiments, the blocks may be performed in a different order than as shown or described in connection with FIG. 5. Such and other variations are contemplated to be within the scope of the present disclosure.

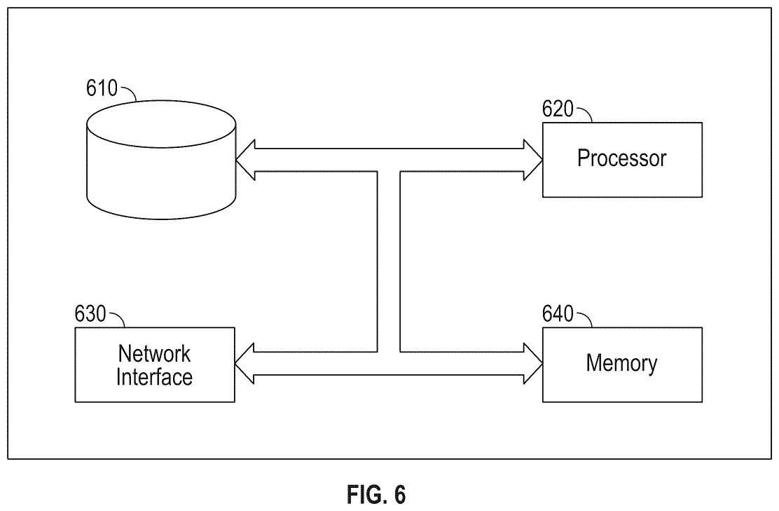

FIG. 6 is a block diagram of an example of computing components that may be used to perform any of the operations or any aspects of the operations described herein, including the aspects and operations described in connection with any of FIGS. 1-5.

The computing components include an electronic storage 610, a processor 620, a memory 640, and a network interface 630. The various components may be communicatively coupled with each other. The processor 620 may be and may include any type of processor, such as a single-core central processing unit (CPU), a multi-core CPU, a microprocessor, a digital signal processor (DSP), a System-on-Chip (SoC), or any other type of processor. The memory 640 may be a volatile type of memory, e.g., RAM, or a non-volatile type of memory, e.g., NAND flash memory. The memory 640 includes processor-readable instructions that are executable by the processor 620 to cause the system to perform various operations, including those mentioned herein, such as the operations described in connection with of FIGS. 1-5.

The electronic storage 610 may be and include any type of electronic storage used for storing data, such as hard disk drive, solid state drive, and/or optical disc, among other types of electronic storage. The electronic storage 610 stores processor-readable instructions for causing the system to perform its operations and stores data associated with such operations, such as storing data relating to any of the sequences, clusters, or confidence scores, among other data. The electronic storage 610 may be a non-transitory processor readable medium. The network interface 630 may implement networking technologies, such as Ethernet, Wi-Fi, and/or other wireless networking technologies.

The components shown in FIG. 6 are merely examples, and persons skilled in the art will understand that a system includes other components not illustrated and may include multiples of any of the illustrated components. Such and other embodiments are contemplated to be within the scope of the present disclosure.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The systems, devices, and/or servers described herein may utilize one or more processors to receive various information and transform the received information to generate an output. The processors may include any type of computing device, computational circuit, or any type of controller or processing circuit capable of executing a series of instructions that are stored in a memory. The processor may include multiple processors and/or multicore central processing units (CPUs) and may include any type of device, such as a microprocessor, graphics processing unit (GPU), digital signal processor, microcontroller, programmable logic device (PLD), field programmable gate array (FPGA), or the like. The processor may also include a memory to store data and/or instructions that, when executed by the one or more processors, causes the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed:

1. A polypeptide sequencing method comprising:
dividing a sample comprising a mixture of polypeptides into at least two factions, the at least two fractions comprising a starting fraction and at least one other fraction;
contacting polypeptides in each of the at least one other fraction with at least one agent for cleavage of the polypeptides within the at least one other fraction, to provide at least one fragmented fraction;
analyzing polypeptides in the starting fraction and polypeptides in the at least one the fragmented fraction to provide starting sequences for polypeptides in the starting fraction and fragment sequences for polypeptides in the at least one fragmented fraction;
grouping the starting sequences and the fragment sequences into at least one cluster, each of the at least one cluster comprising at least one starting sequence of the starting sequences and at least one fragment sequence of the fragment sequences; and
for each cluster of the at least one cluster, generating a computed sequence for the respective cluster based on values in the at least one starting sequence and values in the at least one fragment sequence of the respective cluster, the computed sequence serving as a computationally determined sequence for a polypeptide in the sample.

2. The method of claim 1, wherein the sample is derived from a source selected from the group consisting of: soil, water, air, agricultural, or industrial compositions.

3. The method of claim 1, wherein the sample is a biological sample.

4. The method of claim 3, wherein the biological sample is derived from an animal species with an immune system that produces molecules to defend against infections.

5. The method of claim 1, wherein the mixture of polypeptides in the sample are enriched based on size, charge, or affinity chromatography.

6. The method of claim 1, wherein the cleavage of the polypeptides is achieved enzymatically or chemically.

7. The method of claim 6, wherein at least one agent for cleavage of the polypeptides comprises proteolytic enzymes.

8. The method of claim 1, wherein the starting fraction comprises intact native polypeptides.

9. The method of claim 1, wherein the grouping the starting sequences and the fragment sequences into at least one cluster is based on applying at least one clustering criterion, the at least one clustering criterion applied to at least one of: comparison of two starting sequences, comparison of two fragment sequences, or comparison of a starting sequence with a fragment sequence.

10. The method of claim 1, wherein the at least one clustering criterion comprises at least one criterion using confidence scores for the starting sequences and confidences scores for the fragment sequences.

11. The method of claim 1, wherein, for each cluster of the at least one cluster, the generating the computed sequence for the respective cluster comprises:
positionally aligning the at least one starting sequence and the at least one fragment sequence of the respective cluster with each other, to provide aligned sequences having a plurality of aligned positions; and
for each position of the plurality of aligned positions of the aligned sequences, determining a computed value for the respective position based on values of the aligned sequences at the respective position,
wherein the computed values for the plurality of aligned positions form the computed sequence for the respective cluster.

12. The method of claim 11, wherein the positionally aligning the at least one starting sequence and the at least one fragment sequence of the respective cluster with each other uses Gotoh local alignment technique and BLASTP scoring parameters.

13. The method of claim 11, wherein the computed values for the plurality of aligned positions are consensus values.

14. The method of claim 11, wherein the computed values for the plurality of aligned positions are determined further based on confidence scores for values of the aligned sequences at the plurality of aligned positions.

15. The method of claim 1, further comprising, for each cluster of the at least one cluster:
iterating, for at least one iteration, the generating the computed sequence for the respective cluster, wherein for each iteration, the generated computed sequence from the prior iteration is used as a starting sequence of the respective iteration.

16. The method of claim 1, wherein the analyzing the polypeptides in the starting fraction and the polypeptides in the at least one the fragmented fraction comprises analyzing the polypeptides in the starting fraction and the polypeptides in the at least one the fragmented fraction using tandem mass spectrometry.

17. The method of claim 1, wherein the generating the computed sequence for the respective cluster comprises generating the computed sequence for the respective cluster using a machine learning model

18. The method of claim 17, wherein the machine learning model is a sequence to sequence model.

19. A system for polypeptide sequencing of a sample containing a mixture of polypeptides divided into at least two factions, the at least two fractions comprising a starting fraction and at least one other fraction, where polypeptides in each of the at least one other fraction are contacted with at least one agent for cleavage of the polypeptides within the at least one other fraction to provide at least one fragmented fraction, the system comprising:
at least one processor; and
at least one memory storing instructions which, when executed by the at least one processor, causes the system at least to perform the method of claim 1.

20. A processor-readable medium storing instructions for polypeptide sequencing of a sample containing a mixture of polypeptides divided into at least two factions, the at least two fractions comprising a starting fraction and at least one other fraction, where polypeptides in each of the at least one other fraction are contacted with at least one agent for cleavage of the polypeptides within the at least one other fraction to provide at least one fragmented fraction, wherein the instructions, when executed by at least one processor of a system, cause the system to at least perform the method of claim 1.
